(19) United States
(12) Patent Application Publication
(10) Pub. No.: US 2026/0174747 A1
(43) Pub. Date: Jun. 25, 2026

(54) COMPUTER IMPLEMENTED METHODS OF GENERATING DOSE RECOMMENDATIONS

(71) Applicant: National University of Singapore, Singapore (SG)

(72) Inventors: Dean Ho, Singapore (SG); Agata Blasiak, Singapore (SG); Thanh Lan Anh Truong, Singapore (SG); Kirthika Senthil Kumar, Singapore (SG); Wen Jeit Lester Tan, Singapore (SG); Shi Bei Tan, Singapore (SG)

(21) Appl. No.: 19/128,718

(22) PCT Filed: Nov. 14, 2023
(86) PCT No.: PCT/SG2023/050755

§ 371 Date: May 9, 2025

(30) Foreign Application Priority Data
Nov. 14, 2022 (SG) 10202260082S

(51) Int. Cl. A61K 31/485; A61K 31/519; A61K 38/00; G16H 10/60; G16H 20/10; G16H 40/63; G16H 50/20

(52) U.S. Cl. CPC A61K 31/485; A61K 31/519; A61K 38/00; G16H 10/60; G16H 20/10; G16H 40/63; G16H 50/20

(57) ABSTRACT

A computer implemented method of generating a dose recommendation for a subject is described The method comprises: receiving subject data comprising an indication of a drug dose administered to the subject and an indication of a phenotypic response of the subject, the indication of the phenotypic response of the subject comprising an indication of a change in at least one phenotypic marker level; updating a subject profile which models the phenotypic response of the subject to the drug dose; and generating a dose recommendation for the subject from the subject profile.

### TECHNICAL FIELD

The present disclosure relates to computer implemented methods of determining recommended doses for patients. The disclosure provides personalized pharmacological intervention through small-data phenotypic optimization and predictive capabilities.

### BACKGROUND

The clinical team attending to a patient upon a diagnosis is faced with two main questions: what treatment, and at what dose. Clinical trials' results provide the basis for guidance and support for official protocols that clinicians use to base their decisions upon. However, individuals rarely demonstrate the reported response from relevant clinical trials, often the average from a group representing a population or subpopulation.

### SUMMARY

Phenotypic personalized medicine (PPM) finds an appropriate dosing strategy over time based on small data collected exclusively from the treated individual. In this disclosure we incorporated innovative features introduced to CURATE.AI, in the process of the CURATE.AI team's work on clinical trials and retrospective analyses.

According to a first aspect of the present disclosure, a computer implemented method of generating a dose recommendation for a subject is provided. The method comprises: receiving subject data comprising an indication of a drug dose administered to the subject and an indication of a phenotypic response of the subject, the indication of the phenotypic response of the patient comprising an indication of a change in at least one phenotypic marker level; updating a subject profile which models the phenotypic response of the subject to the drug dose; and generating a dose recommendation for the subject from the subject profile.

The subject profile may be generated based on second order relationship, a higher order relationship or a lower order relationship between the phenotypic marker level and the drug dose.

In an embodiment, the dose recommendation for the subject is selected to calibrate the subject profile during a dynamic calibration phase. This provides personalized, dynamic calibration with the following features: Reconciliation of the technical and clinical requirements for a generation of a reliable and actionable profile. Adaptation of the established Design of Experiment (DoE) methods (e.g. D-optimization) with pre-specified clinical considerations, e.g. avoiding extreme doses and avoiding extrapolation in the personalized dosing range. The methods allow for dynamic adaptation of the calibration doses in response to the outcome measures or changes to the subject state. Further, the methods allow for improved performance in the efficacy dosing stage within a narrower dosing range, while outlining the broad dosing landscape for each subject. Embodiments provide agility in adjusting the calibration strategy based on minimal data and/or dynamically changing subject state during the calibration leading to less delays in the transitions to efficacy driven dosing and an ability to incorporate the initial and dynamically changing risk preferences.

The subject data may comprise an indication of at least three drug doses administered to the subject and at least three corresponding phenotypic responses.

In an embodiment, generating the dose recommendation for the subject from the subject profile comprises transforming data from previously received subject data. This provides data transformation for flexible adaptation to various clinical use cases. This unique application of data transformation (e.g. cumulative dose in a given period) for the purpose of accommodating various clinical factors (e.g. adherence pattern, treatment schedules) increases the platform implementation potential for different clinical use cases. This flexible process can also broaden the applicability of the algorithm to a wide range of disease indications. Furthermore, adapting the algorithm to different clinical factors also improves the technical accuracy of the generated profile.

In an embodiment, generating the dose recommendation comprises determining a composite outcome measure which is dependent on a plurality of marker levels. This allows incorporation of multiple outcome measures into the decision making, either by creating a composite outcome measure, or by independent outcome measure analyses, one or more may incorporate the methods described above, and subsequent reconciliation of the recommendations. This enables multifaceted monitoring and guidance for intervening into the subject state.

In an embodiment, the subject profile is based on subject data corresponding to a plurality of different times and the subject data is weighted according to the time such that a higher weighting is given to more recent data points. This provides for incorporation of a time component allowing evolution of the subject profile with a higher weightage given to latest data points. The algorithmic approach takes into consideration evolving conditions with respect to time. In cases when a subject's state or condition undergoes transition with time, the previous or older state becomes less relevant to the current state. Also, in some cases doses accumulate over time in the subject's body. Hence, by incorporating a time component and more weightage to the latest data points the subject's profile can be more accurately and dynamically represented. In turn, aiding in more accurate dosing for the current state.

In an embodiment, the method further comprises receiving clinician inputs corresponding to a decision tree and wherein the dose recommendation is generated using the clinician inputs and the subject profile. This provides an actionable decision tree that incorporates common clinical events into the workflow, and/or prompts for further user inputs. This actionable decision tree enables a holistic and transparent dose optimization approach, which not only incorporates more common clinical events (e.g. acute infection with short-term effect) within the workflow than before, but also prompts for further user actions in the case of practical concerns (e.g. profile unable to recommend a therapeutic dose). The decision tree also verifies whether a clinical event has short-term or prolonged change in subject state through dual-intent dose recommendation (i.e. calibration-intent and efficacy-driven). This new method step enables evolving a current profile which takes shorter time than profile recalibration, thereby benefiting patients with more efficacy-driven dose recommendations. The decision tree also enables human oversight (e.g. deferring dose decision to physician) that serves as a contingency plan when the profile is unable to recommend a dose and when patients require immediate attention (e.g. severe adverse events). This method step establishes a safety net by alerting the users for further actions. Collectively, the new decision tree enables a holistic and safe implementation of the platform for routine clinical use.

In an embodiment, the dose recommendation is generated taking into account patient compliance and/or risk factors. This allows for incorporation of human factors in the dose recommendations, such as patient adherence/compliance to the medication and patient-specific acceptable risk level. Risk level can refer to the technical performance of the methods, e.g. prediction reliability cut off, the extent of extrapolation, or to how far the dose recommendation is from the currently established standard of care (SOC). Adherence of newly recommended doses can be maximized through a behavioral approach, by adjusting the dose recommendations based on the subject's history of pill adherence and inferred likelihood of complying with smaller or larger number of pills. Higher patient compliance due to optimized dose pill counts will facilitate the optimization process and may result in more desirable patient outcomes. Incorporation of the risk preference allows treatment personalization aligned with patient autonomy over health-care decisions. Safety feature that can be tailored to the user's (physician's) preference as aligned with human-in-the-loop principles.

In an embodiment, the method further comprises comparing the subject profile with profiles corresponding to different patients or examination of features such as the shape and position of the subject profile. This allows leveraging of the predictive potential of the generated profiles. The initial profile may reveal unexpected drug interactions (e.g. synergistic, antagonistic interactions) at certain drug/dose ranges in drug combinations, which enables selecting doses with beneficial interactions. Evolving profiles subsequently predict treatment outcomes over time, which uniquely enables prioritization and de-prioritization of therapies. For instance, a profile completely outside the therapeutic range may predict that the current regimen will not be effective, and an alternative regimen may be prioritized.

In an embodiment, outcome measures are used to evaluate the technical performance and clinical relevance of the method. In some embodiments of analysis, decision to evaluate method effectiveness considers logistical and practical feasibility factors. The novel outcome measures devised behave as a proxy metric to evaluate the platform's performance. It also behaves as evidence comparable to the clinically utilized metrics aiding in decision tree on method selection.

In an embodiment, the dose recommendation is selected to achieve a change in marker level within a target range. Dose recommendation may be tailored to the desired size effect of the change within the predictable output range. E.g., the method can recommend the dose resulting in predetermined percent change of the response marker in contrast to predicting a directional change in the response marker level. Controlling the change in response marker to tailor it to the treatment plan, e.g. slowing down disease response in accordance with the evolutionary-based adaptive dosing in oncology to delay patient relapse, or to e.g. avoid side effects of the rapid change in the patient state.

Embodiments of the method provide high resolution dose selection for subset of subjects. In traditional clinical scenarios, some subjects achieve a wide response range with a narrow dosing range. This response range also comprises of the relevant therapeutic range. In those cases, it becomes challenging to identify the right dose within the narrow dosing range. With the technical capabilities of this invention, the resolution of the doses can be improved aiding in choosing the exact dose.

In an embodiment, the method further comprises selecting an algorithm from a plurality of algorithms for updating the subject profile. Selection and switching between algorithmic variants can be achieved by a ranking system that takes into consideration both technical and clinical parameters and assists in selection of the most optimal algorithmic variant for a specific indication and time point: e.g. cumulative, rolling window or dynamic shift, and switching between each other.

According to a second aspect of the present disclosure, a data processing system configured to carry out a method set out above is provided.

According to a third aspect of the present disclosure, a computer readable carrier medium carrying processor executable instructions which when executed on a processor cause the processor to carry out a method as set out above is provided.

The methods of the present disclosure rely on rapid and reliable biomarkers that should accurately reflect the intervention's effects. The undetected patient non-adherence to the treatment and an analytical error in the biomarker measurement may lead to inaccuracies in the performance, therefore, indications for deployment are limited to those where patient adherence can be detected, and a reliable biomarker is available.

Additionally, the biomarker should be minimally invasive to allow for longitudinal monitoring and dynamic adjustments.

### BRIEF DESCRIPTION OF THE DRAWINGS

In the following, embodiments of the present invention will be described as non-limiting examples with reference to the accompanying drawings in which:

FIG. 1 shows an overview of a dose recommendation method according to an embodiment of the present invention;

FIG. 2 is a block diagram showing functional modules of a dose recommendation system according to an embodiment of the present invention;

FIG. 3 is a flow diagram showing a method of generating dose recommendations according to an embodiment of the present invention;

FIG. 4A to FIG. 4C are graphs showing doses for different patients calculated using methods according to embodiments of the present invention;

FIG. 5 is a graph showing dynamic evolution of a patient profile;

FIG. 6A to FIG. 6D are tables and graphs showing simulated doses and patient profile evolutions;

FIG. 7 is a flowchart showing the determination of whether predictions of changes in a target marker were successful;

FIG. 8 is a flowchart showing validation of methods of dose recommendation;

FIG. 9 is a chart showing Serial Percentage Absolute Prediction Error (PAPE) for dose recommendation methods;

FIG. 10A and FIG. 10B are graphs comparing linear and quadratic methods;

FIG. 11A to FIG. 11D are graphs a comparison of dosing recommendations generated using methods of embodiments of the present invention with physician guided dosing;

FIG. 12A to FIG. 12C are graphs showing comparisons of quadratic and linear methods;

FIG. 13 shows an application of the dose recommendation system to a mobile health platform;

FIG. 14 illustrates a retrospective analysis of pediatric liver transplant data;

FIG. 15 shows TTL over time of each patient;

FIG. 16 shows a treatment journey for a patient with and without CURATE.AI;

FIG. 17 shows CURATE.AI dose modulations to achieve and sustain the therapeutic range; and

FIG. 18 is a block diagram showing a technical architecture of a data processing system according to an embodiment of the present invention.

### DETAILED DESCRIPTION

The present disclosure provides a platform for dose recommendation that is mechanism agnostic and disease independent.

For combinatorial drugs, the therapeutic response of a patient to drug doses is modeled using the following equation.

$$E ( t ) = {E}_{0} + {\sum }_{i} {a}_{i} {x}_{i} ( t ) + {\sum }_{{ii}^{′}} {b}_{{ii}^{′}} {x}_{i} ( t ) {x}_{{i}^{′}} ( t ) + higher order terms ( optional )$$

where E(t) is the time-varying therapeutic response (either an independent outcome measure or composite outcome measure) for a patient at time t, E₀ is a parameter or constant corresponding to a baseline therapeutic outcome for the patient, xᵢ(t) is a time-varying dose or concentration for the patient of an i^{th} drug at time t, and may also be cumulative doses or concentrations when suitable. aᵢ is a parameter or constant corresponding to a second order transfer function between the therapeutic outcome and the i^{th} and i′^{th} drugs representing drug-drug interaction, and the summations run through corresponding to the total number of drugs in a drug combination being evaluated. The therapeutic outcome is typically represented by a low order equation, such as second order (quadratic) equation, but a first order (linear) equation as well as a third order (cubic) equation may be possible as well. Higher order equations may also be possible for other embodiments. Importantly, only the patient's own data that are prospectively acquired, or retrospectively analyzed, instead of big data from a population, are used to recommend the next dose for that patient.

In another embodiment xᵢ is time independent, e.g. the dose-response data are collected in the same time, based on independent, localized stimuli and response collection, e.g. localized skin treatment. Another example would be a situation when patient sample is treated ex-vivo with drugs at a number of dose levels matching the mathematical requirement of the applied equation order and number of drugs in the regimen.

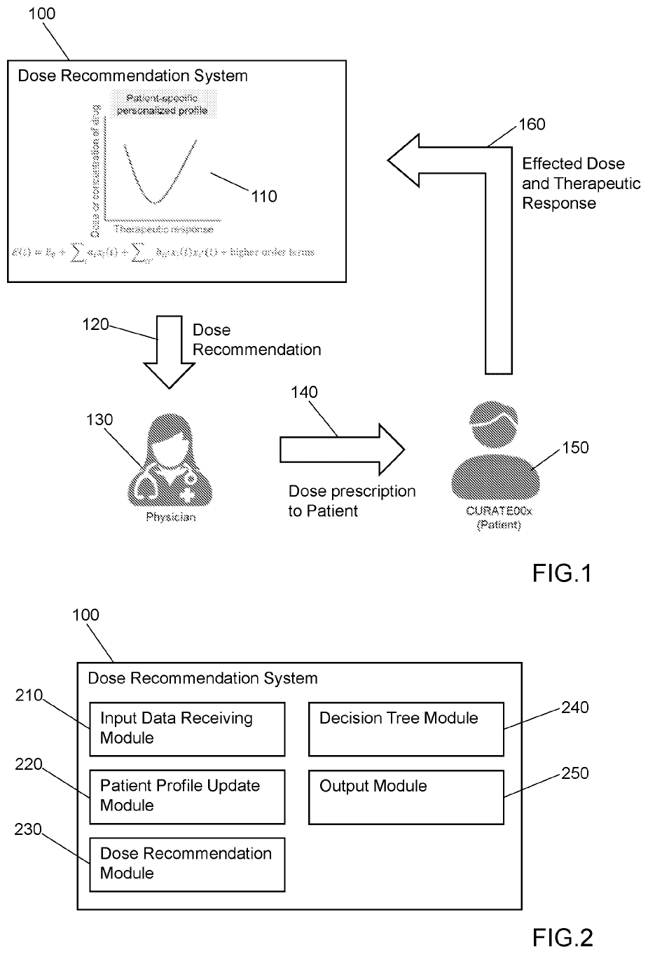

FIG. 1 shows an overview of a dose recommendation method according to an embodiment of the present invention. As shown in FIG. 1, a dose recommendation system 100 uses a patient specific profile 110 to provide a dose recommendation 120 to a physician 130. The physician 130 then prescribes a dose 140 to the patient 150. Following the patient 150 taking the drug dose, data 160 indicating the effected dose and the patient therapeutic dose is input into the dose recommendation system 100. The dose recommendation system 100 uses the data 160 to update the patient profile 110. A shown in FIG. 1, data is collected and integrated into the dose recommendation dynamically. Only the individual patient data are used to generate dose recommendation for that patient for the next dosing cycle. New generated data are included in the patient specific, personalized profiles, such that the patient profile evolves with the patient journey.

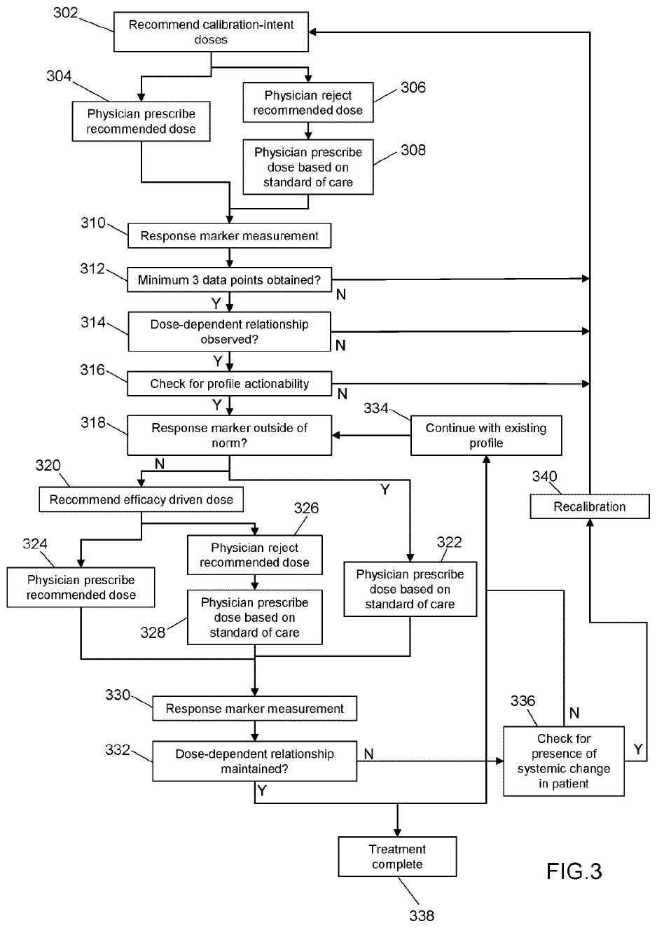

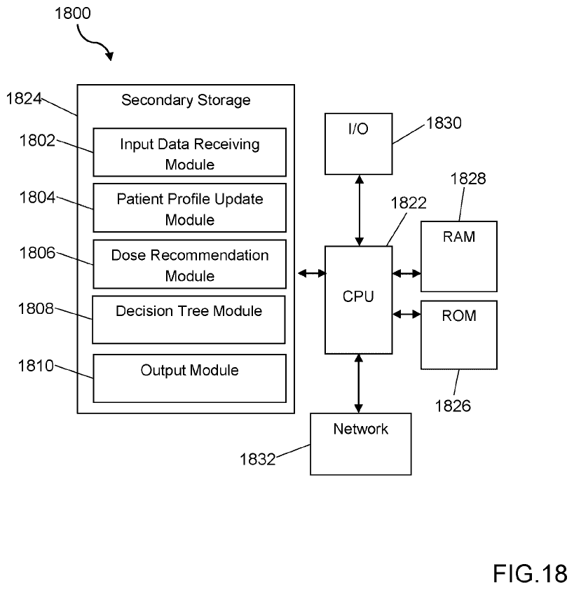

FIG. 2 is a block diagram showing functional modules of a dose recommendation system according to an embodiment of the present invention. The dose recommendation system may be a computer system comprising hardware and software which implements the functional modules shown in FIG. 2. A detailed technical architecture of the dose recommendation is described below with reference to FIG. 18. As shown in FIG. 2, the dose recommendation system 100 comprises an input data receiving module 210, a patient profile update module 220, a dose recommendation module 230, a decision tree module 240 and an output module 250. The input data receiving module 210 receives data indicating effected dose and therapeutic response for a patient. The input data may be manually input, for example using a keyboard or other input device, or may be received from a sensing device or remote device. The patient profile update module 220 updates the patient profile for the patient based on the received effected dose and therapeutic response. The dose recommendation module 230 uses the patient profile to generate a dose recommendation for the patient. As will be described in more detail below, the dose recommendation system 100 may generate dose recommendations with either a calibration intent or an efficacy driven intent. The decision tree module 240 follows a decision tree such as that shown in FIG. 3 to determine how the dose recommendations are generated. The decision tree module 240 may determine whether the dose recommendations are generated with a calibration intent or an efficacy driven intent. The output module 250 generates output indications of dose recommendations which are used by the physician to prescribe doses for the patient.

FIG. 3 shows a method of generating dose recommendations according to an embodiment of the present invention. The method shown in FIG. 3 is carried out by the dose recommendation system 100 shown in FIG. 2. The method demonstrates the calibration-intent and the efficacy-driven dose recommendation stages. Of note, elements of this workflow may be adapted to a specific indication, e.g. by adding additional stoppage criteria or adding/removing additional physician oversight steps.

In step 302, the dose recommendation system 100 recommends calibration intent doses. An initial dose or set of doses is generated to calibrate the patient profile. The dose recommendations are provided to the physician and the physician decides on whether to accept the dose recommendations or not. If the physician accepts the doses, the method moves to step 304 in which the physician prescribes the recommended dose(s) to the patient. If the clinician rejects the recommended dose, the method moves to step 306 and in step 308, the clinician prescribes the dose based on standard of care. In step 310, following the patient taking the prescribed dose, the response marker measurement is carried out and the dose recommendation system 100 receives data indicating the effected dose and therapeutic response. This data is added to the patient profile and then in step 312 it is determined whether a minimum of three marker points have been obtained. If not, the method goes back to step 302 and further calibration intent doses are generated. If three or more marker points have been obtained, the method moves to step 314 in which the patient profile is examined to determine if a dose dependent relationship has been observed. If no dose dependent relationship is observed, the method returns to step 302 and further calibration intent doses are generated. If a dose dependent relationship is observed, the method moves to step 316 in which the patient profile is checked for actionability. If the patient profile is actionable, the method moves to step 318, if not the method returns to step 302 and further calibration intent doses are generated.

In step 318, the efficacy based recommendation begins. The patient profile is used to generate a dose recommendation and in step 318 it is checked whether the dose recommendation is outside the norm. If the dose recommendation is within the norm, the efficacy driven dose is provided to the physician as a recommended dose in step 320. If the response marker is outside the norm the clinician prescribes a dose based on standard of care. Following step 320, the physician may either prescribe the recommended dose in step 324 or reject the recommended dose in step 326. If the physician rejects the recommended dose, the physician will prescribe a dose based on standard of care in step 328.

Following step 322, 324 or 328, the patient takes the recommended dose and in step 330, marker measurement is carried out and the dose recommendation system 100 receives data indicating the effected dose and therapeutic response. This data is added to the patient profile.

In step 332 it is determined by the dose recommendation system 100 whether the dose dependent relationship is maintained. If the relationship is maintained, the method returns to step 334 and the process continues with the existing patient profile. Alternatively, if the treatment is completed, the method moves to step 338.

If the dose dependent relationship is not maintained, the method moves to step 336 in which a check is made for a systemic change in the patient. If there is no systemic change in the patient, the method moves to step 334 and the process continues with the existing patient profile. If there is a systemic change in the patient, the method moves to step 340 in which recalibration is carried out. The recalibration may include creation of a new patient profile or an adjustment of the existing patient profile.

As described above, the method provides dose recommendations at two stages, characterized by their intent. First, dose recommendations have an intent of dynamic calibration and building patient profile, based on a minimal prospectively collected dataset. The doses recommended by the dose recommendation system 100 with calibration intent may rely on adapting design of experiment (DoE) methods (e.g. D-optimization) to clinical considerations, including avoiding extreme doses and minimizing extrapolation within the dosing range. This dynamic adaptation of the calibration doses also considers the patient response or changes to the patient state.

To dynamically adapt to the doses and responses over time for dose recommendations, the dose recommendation system 100 uses a few algorithmic variations including but not limited to: the cumulative approach where the doses and responses from the chosen baseline are used, the rolling window approach where the most recent three (for second order equation) or two (for first order equation) sets of dose-response pairs with unique doses from the timepoint that requires a dose recommendation are used, and the dynamic shift where the generated profiles based on the equation at a particular timepoint is shifted vertically based on the prediction error (the difference between the observed and predicted response based on the equation) of the previous timepoint. Other algorithmic variants that use the approach of the second order or first order equation, or higher order equations, to correlate the responses to doses may be possible as well. Novel outcome measures are used as a proxy metric to evaluate the chosen algorithmic variant to inform potential improvements or disease progression. The variants are ranked in consideration of both technical and clinical parameters to assist in the selection of the most optimal variants.

The second order or first order equation, or higher order equation, that correlate the doses and responses of a patient over time is used to generate a personalized patient profile. The dose recommendation system 100 will recommend a specific dose corresponding to the target response (e.g. desired size effect of the change within the predictable output range). A recommendation is available only when the dose corresponding to the target response is within the x-axis limits, defined as the dosing range determined by physicians and when the target response falls within the personalized patient profile generated. Extrapolation of the patient profile beyond existing data is minimized.

Over time, the personalized patient profile may evolve by allocating more weightage to the latest data points in the patient profile, in cases when a patient's state or condition undergoes transition with time which results in the previous or older state becoming less relevant to the current state, or when doses accumulate over time in the patient's body.

The dose recommended is in accordance with an actionable decision tree that incorporates common clinical events and prompts for further user actions in the case of practical concerns, and considers human factors (e.g. patient adherence to the medication and patient-specific acceptable risk level).

The patient profile may reveal unexpected drug interactions (e.g. synergistic, antagonistic interactions) at certain drug/dose ranges in drug combinations, which enables selecting doses with beneficial interactions. Evolving profiles subsequently predict treatment outcomes over time, which uniquely enables prioritization and de-prioritization of therapies. For instance, a profile completely outside the therapeutic range may predict that the current regimen will not be effective, and an alternative regimen may be prioritized.

Studies with CURATE.AI

The following studies are based on several retrospective analysis and prospective studies.

### 1. Precise CURATE.AI Trial, Solid Tumor Cohort

CURATE.AI is a mechanism agnostic platform that enables dynamic generation of personalized dose recommendations based on a quadratic relationship between intervention intensity (e.g. drug dose) and phenotypic output (e.g. tumor marker). The input data used to generate a personalized CURATE.AI recommendation consisted exclusively of the data of the patient whose doses were being optimized as align with algorithmic fairness principles. The data used to construct the quadratic relationship were: capecitabine dose taken (the prescribed dose adjusted by the pharmacovigilance information) and the resulting marker level change compared to the previous cycle. The incurred side effects, the actual doses and the marker levels in the previous cycles, patient and user adherence to the treatment and the dose recommendations, respectively, were incorporated in the interpretation of the algorithmic results and included in the recommendation provided to the users. After the calibration stage was completed and the profile was generated the dosing intent changed from ‘calibration intent’ to ‘efficacy driven’. The dose-response data pairs for the subsequent cycles were incorporated into the profile at each dosing event, such that the profile and the CURATE.AI recommendations based on the profile dynamically evolved along the patient's journey.

The users of CURATE.AI recommendations were treating physicians engaged as Co-Investigator in this study. They were introduced to the principles of CURATE.AI during site initiation visit (SIV). Although no formal training was provided, they had additional opportunities to familiarize themselves with CURATE.AI during ground rounds and seminars as well as literature. The CURATE.AI output was presented to the users at each dosing event via email as a recommendation sheet that included the dose recommendation and the recommendation intent information. The users were free to ask additional questions to the CURATE.AI team.

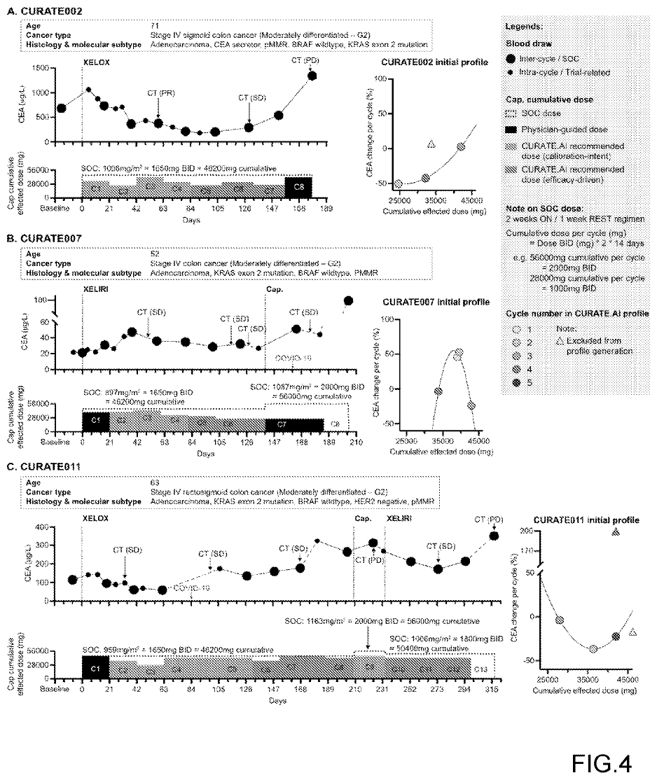

FIG. 4A to FIG. 4C show doses for different patients calculated using the CURATE.AI system.

The dose recommendations by CURATE.AI formed personalized calibration-see the difference in dose recommendations for patients CURATE002, CURATE007 and CURATE011.

Adaptability of the approach to use cumulative dose taken over the dosing cycle or prespecified period of time allowed to incorporate unexpected dosing breaks into the dose recommendation procedure (see FIG. 4)

The outcome measure (CEA marker) can be expanded to include a composite measure with another efficacy marker (e. g. CT scans) or toxicity markers (blood results) (see FIG. 4A, B, C).

Physician-guided and recalibration strategies (FIG. 4 A, B, C) at the later stages of patient journey represent the outcomes of the decision tree allowing flexibility of the approach and adjusting to the clinical circumstances encountered in real world, such that CURATE.AI can serve not only ‘the perfect patient’ of clinical trials.

CURATE.AI recommendation from the upfront includes safety features like dose recommendations only in-between 50-100% SOC. This range can be further dynamically adjusted by the physician to reflect their or their patient risk preference. CURATE.AI This can be done for a specific dosing event or included systematically into the algorithm.

The profiles (FIG. 4 A, B, C) describe the expected result for the whole tested dosing range. As such, the clinical decision may be supported not only for the maximum effects, but also for intermediate effects, e.g. to control temporal dynamics of tumor shrinkage. It may facilitate guidance of approaches like evolutionary dosing.

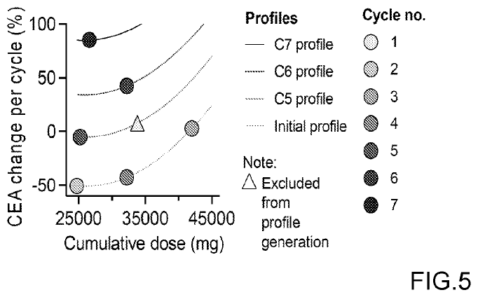

FIG. 5 shows the dynamically evolving profile of patient CURATE002. The shift which starts at cycle 5 points out to relapse by cycle 7.

The profile shape informs about the possible marker outcomes and can be used as a predictive marker of the given regimen to aid decision on its suitability, e.g. profile that demonstrates that no dose will result in a marker drop indicates the regimen is not suitable in the specified dosing range. This understanding can support prognostic abilities to inform about the likely patient overall outcome. Marker response that is significantly misaligned with the prediction indicates systemic change and may inform about relapse.

### 2. Warfarin CURATE.AI Retrospective Analysis

### 2.1 Data Collection

This retrospective study was approved by the National Healthcare Group Domain Specific Review Board, NHG DSRB 2020/00019. The study analyzed the clinical records of 127 patients who underwent warfarin therapy in the year of 2018 in the National University Hospital (NUH), a tertiary hospital in Singapore. De-identified data containing the patient demographics, prescribed warfarin doses, corresponding observed INR and date of INR measurements was obtained for the analysis from Discovery.AI, a platform that collates NUH patient data in an anonymized fashion.

As this current study aimed to demonstrate CURATE.AI applicability for warfarin dosing, patient records that had sufficient dose-response data to generate at least one second-order dose-response profile and were able to generate a minimum of one INR prediction were identified. Data on genetic factors were not required in the CURATE.AI dosing optimization process and were not collected.

### 2.2 Personalized Dosing Optimization Process with CURATE.AI

The process of CURATE.AI dose optimization using second order and first order functions as the backbone of CURATE.AI (CURATE.AI Quadratic and CURATE.AI Linear, respectively) is demonstrated below using simulated patient data.

### 2.2.1 Simulated CURATE.AI Quadratic Process

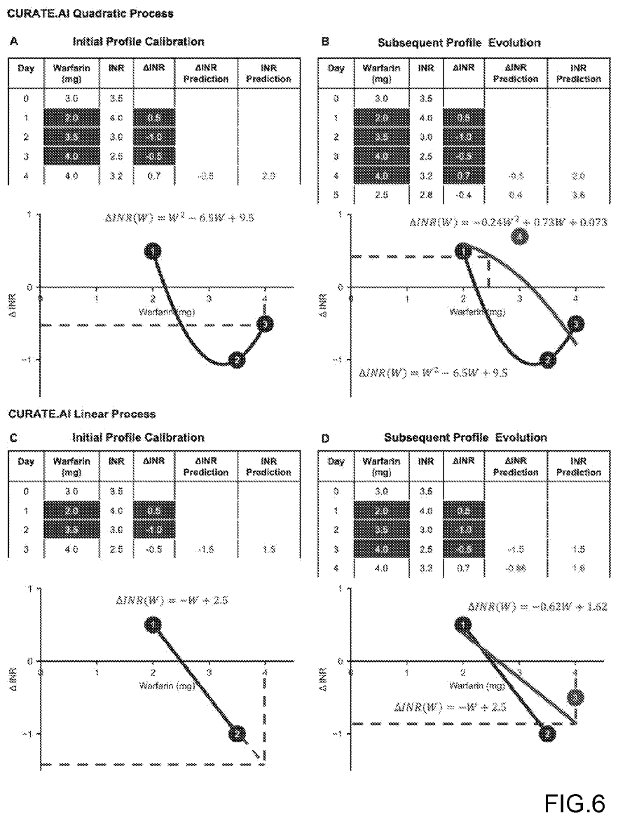

FIG. 6A to FIG. 6D show CURATE.AI Quadratic and Linear Process with Simulated Data. Simulated records of patient's prescribed warfarin doses and their corresponding INR measurements were used. CURATE.AI Quadratic (A and B): The patient's initial profile (blue) was calibrated using dose-response data pairs on days 1 to 3. The profile was used to predict the patient's ΔINR and INR response to the warfarin dose given on day 4. Numbers within the circles correspond to the dosing days with the given warfarin dose. (A) Initial calibrated profile (blue) using the dose-response data pairs from days 1 to 3. (B) Assuming no systemic and regimen changes, the profile evolves to include the observed dose-response data pair on day 4. CURATE.AI Linear (C and D): The patient's initial profile (blue) was calibrated using dose response data pairs on days 1 and 2. The profile was used to predict the patient's ΔINR and INR response to the warfarin dose given on day 3. Numbers within the circles correspond to the dosing days with the given warfarin dose. (C) Initial calibrated profile (blue) using the dose-response data pairs from days 1 and 2. (D) With no systemic and regimen changes, the profile evolves to include the observed dose-response data pair on day 3.

FIGS. 6A and 6B demonstrate the CURATE.AI Quadratic dosing optimization process using simulated data. In this simulation, warfarin dose was modulated against the change in the phenotypic output, change in INR between two consecutive days (ΔINR).

With a minimum of 3 uniquely modulated doses, the administered warfarin dose (input), represented by W, and the corresponding ΔINR response (output) from days 1, 2 and 3 were mapped via a second order response equation ΔINR (W)=W²−6.5 W+9.5 (FIG. 6A) where the equation coefficients [1; −0.65; 9.5] were specific to the simulated case at the given timepoint. This equation represents the patient's profile. The ΔINR and INR predictions for day 4 are computed from this initial profile with the next recorded dose of 4 mg.

With no systemic or regimen changes, the observed dose-response data pair on day 4 was included into the patient's profile, represented by the evolved response equation INR (W)=−0.24W²+0.73 W+0.073 (FIG. 6B) with new coefficients [−0.24; 0.73; 0.073] specific to the simulated case at the new timepoint. Predictions for ΔINR and INR for day 5 are computed and the process is repeated for the remaining timepoints.

### 2.2.2 Simulated CURATE.AI Linear Process

The CURATE.AI Linear process is similar to that of CURATE.AI Quadratic. It requires a minimum of 2, instead of 3 uniquely modulated doses and the corresponding phenotypic output to calibrate a profile. The modulated warfarin doses and ΔINR response from days 1 and 2 are mapped via a first order response equation represented by ΔINR(W)=−W+2.5 (FIG. 4C). From this initial calibrated profile and the next recorded dose of 4 mg, the predictions for ΔINR and INR for day 3 are computed.

With no systemic or regimen changes on day 3, the observed dose response data pair on day 3 is included in the profile, yielding a response equation INR (W)=−0.62 W+1.62 (FIG. 4D). Predictions for ΔINR and INR for day 4 are computed and the process repeats for the remaining timepoints.

### 2.3 Evaluation of CURATE.AI's Predictive Performance

In order to evaluate CURATE.AI's predictive performance, six metrics were identified: three technical performance metrics and three clinically relevant performance metrics (Table 1).

TABLE 1
Table of Metrics.

| Group | Metrics | Formula |
|---|---|---|
| Technical Performance | Percentage Absolute | $\frac{abs ( Predicted INR - Observed INR )}{Observed INR} \%$ |
| Metrics | Prediction Error | |
| | (PAPE) | |
| | Percentage Prediction Error | $\frac{Predicted INR - Observed INR}{Observed INR} \%$ |
| | (PPE) | |
| | PPE 20% | −20% < PPE < 20% |
| Clinical | INR Clinical | lower bound < Predicted INR − |
| Relevant | Prediction Error | Observed INR < upper bound |
| Performance | (CPE) | |
| Metrics | Underprediction | Predicted INR − Observed INR < 0 |
| | Bias | |
| | Modelled | |
| | Potential Time in Therapeutic | $\frac{No . of prediction events marked as success}{Total no . of predictions made} \%$ |
| | Range (TTR) | |

### 2.3.1 Technical Performance Metrics

Three metrics were selected from literature review to perform cross model comparison for models used for serial INR prediction. Percentage absolute prediction error (PAPE) was selected from Vadher et al's population-based pharmacokinetic/pharmacodynamic (PKPD) model. The model uses an empirical model which describes the relationship between the vitamin K dependent clotting factors and the INR, and the Bayesian method for parameter estimation. CURATE.AI's PAPE was compared with Vadher's Bayesian PKPD model by identifying patients with sufficient data for at least four predictions to match the number of predictions between the two models. Additionally, five-fold cross validation was employed for the evaluation of PAPE for CURATE.AI Quadratic and CURATE.AI Linear, to test their generalizability to other independent data sets with similar underlying patient characteristics. This test may also inform of the CURATE.AI predictive models being subject to bias from the variability of the underlying data set.

The second selected technical metric was percentage prediction error (PPE) to benchmark the CURATE.AI performance against the performance of Xue et al's dose-response model, Xue et al's PKPD model and Hamberg et al's PKPD model reported for an independent dataset. The mean/median percentage PE for those models was obtained by summing each category across the five hospitals' data they were externally evaluated against. Of note, all those three models require population-based estimates of parameters alongside patient specific parameters like genetic makeup, observed warfarin dose and INR response, while CURATE.AI requires 2 to 3 longitudinal dose-response data pairs of the treated patient only to generate its first recommendation. We also selected INR percentage prediction error of 20% (PPE 20%) from Xue et al's model evaluation. PPE 20% considers INR prediction error within +20% as ideal. INR predictions with an error <−20% and >20% are considered as underprediction and overprediction, respectively.

### 2.3.2 Clinically Relevant Performance Metrics

In addition to the existing metrics available in the literature to evaluate models used for serial INR prediction, three metrics were newly devised to assess the clinical relevance of CURATE.AI (Table 1). A literature review revealed a lack of clinically relevant metrics to evaluate serial INR prediction performance. Given that the goal of CURATE.AI is to be deployed as a CDSS in the prospective management of warfarin dosing, it is essential that clinically relevant metrics are part of the criteria for performance evaluation.

The first proposed metric—INR clinical prediction error (CPE)—assessed the percentage of the predictions whose CPE was within prespecified, clinically-motivated error threshold. INR therapeutic range is 2.0-3.0. Given that the risk of bleeding increases significantly with INR>5.0 and risk of thromboembolism with INR<1.5 [50], we considered 2.0 and 0.5 error thresholds as clinically relevant and included them in the CURATE.AI performance assessment.

The second proposed metric-underprediction bias-focused on negative prediction error. Underprediction bias increases the risk of overdosing warfarin, in turn increasing the patient's risk of bleeding that is not easily reversed.

The third proposed metric—modelled potential time in therapeutic range (TTR)—was generated to estimate CURATE.AI's capability to support warfarin dosing in order to achieve an INR within the therapeutic range for the maximum time. Each prediction instance was labelled as success or non-success (FIG. 5). To be labelled as success—leading to INR within therapeutic range—the dosing instance had to fulfil the following:
1. Both the corresponding predicted INR and the observed INR were in therapeutic range—CURATE.AI correctly predicted the INR to be in target range
2. Predicted INR and the observed INR were both below or both above the therapeutic range but there exist an actionable dose—a dose that CURATE.AI would have potentially recommend that lies within 0 to 14 mg and is not the observed dose, that would achieve target INR.

The CURATE.AI modelled potential TTR for a single patient was then calculated by the percentage of success instances over the total number of predictions. This modelled potential TTR was compared against physician-guided TTR which was calculated by the percentage of prediction events resulting in an INR in the therapeutic range over the total number of INR measurements, beginning from the first CURATE.AI prediction.

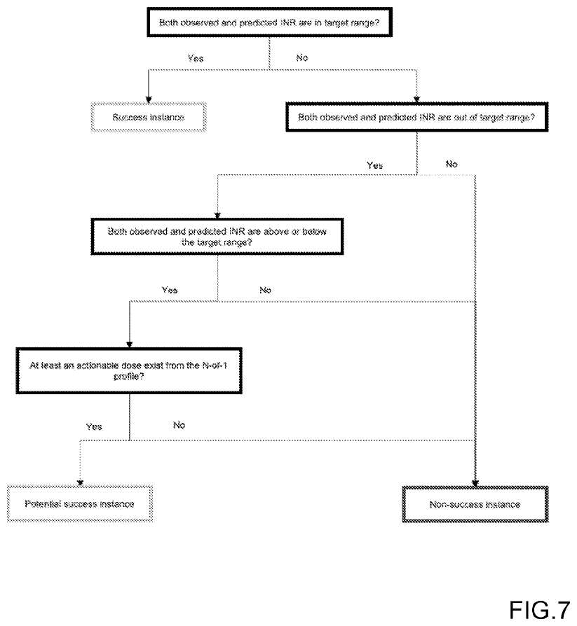

FIG. 7 shows a Decision Flowchart of Modelled Potential TTR. Each prediction instance is first determined if they count as a success instance using an algorithm.

### 2.4. Statistical Analysis

Data analysis was performed using scripts written in Python (Version 3.8.0). Normality test was performed with Shapiro-Wilk test at α=0.05. Statistical comparison were performed with Wilcoxon signed-rank test at α=0.05.

### 2.5. Results

### 2.5.1. Patient Population

The study analyzed the clinical records of 127 patients who underwent warfarin therapy for deep venous thrombosis, pulmonary embolism and atrial fibrillation in the year of 2018 in the National University Hospital (NUH). The demographic characteristics of these patients are listed in Table 2.

TABLE 2
Patient demographics. Mean [range]

| | Patients |
|---|---|
| Variables | (N = 127) |
| Sex | |
| Male | 76 (59.8%) |
| Female | 51 (40.2%) |
| Age [years] | 58.5 [18-99]^{a)} |
| Ethnicity | |
| Chinese | 83 |
| Malay | 18 |
| Indian | 10 |
| Others | 16 |

As this current study aimed to demonstrate CURATE.AI's applicability in warfarin dosing, all patients who had sufficient warfarin dose-ΔINR data pairs to generate a CURATE.AI profile and to generate a minimum of one INR prediction were identified.

A total of N=92 patients and N=118 patients were identified to have met the requirement for CURATE.AI Quadratic and CURATE.AI Linear analysis, respectively (FIG. 6). The target range of INR was assumed 2-3 for all patients.

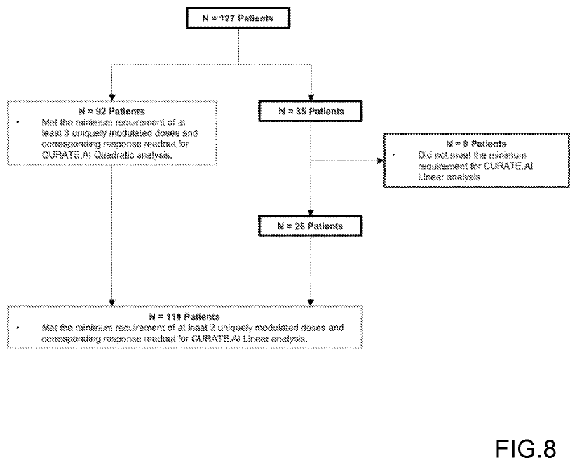

FIG. 8 is a flowchart showing Internal Validation Data Screening Flow. A total of N=127 de-identified patient data were retrieved from the Discovery.AI Platform. N=92 and N=118 patient data met the requirements of a minimum of 3 and 2 modulated doses and corresponding response readout for CURATE.AI Quadratic and CURATE.AI Linear profile analysis, respectively. N=9 patient data did not meet the requirement and were excluded from the analysis. N=number of patients.

### 2.5.2 Out-of-Bounds (OOB) Predictions

It is possible for models to generate predictions outside of physiological and clinical quantitation means. Out-of-Bounds (OOB) predictions refer to INR predictions that are impossible in the physiological context (negative INR) and values beyond the limit of quantitation (INR>15). As CURATE.AI is intended as CDSS with physician oversight, the OOB predictions could be actively discarded should they occur. 1.15% (n=9 out of 786 predictions) of INR predictions from CURATE.AI Quadratic and 0.88% (n=9 out of 1025 predictions) of INR predictions from CURATE.AI Linear model were considered OOB and removed from the subsequent analyses.

### 2.5.3 CURATE.AI Technical Performance Metrics

### 2.5.3.1 Percentage Absolute Prediction Error (PAPE)

The median PAPE across all predictions for CURATE.AI Quadratic and CURATE.AI Linear are shown in Table 3.

TABLE 3
CURATE.AI PAPE. Values are reported as median [95% CI].
n = total number of prediction events.

| | PAPE |
|---|---|
| Model | Median [95% CI] |
| CURATE.AI Quadratic | 14.9 |
| (n = 777) | [13.7-17.0] |
| CURATE.AI Linear | 14.3 |
| (n = 1016) | [13.3-15.5] |

To compare CURATE.AI's performance with Vadher's Bayesian PKPD model, patients with sufficient data for at least four predictions were identified for this analysis (CURATE.AI Quadratic: N=62, CURATE.AI Linear: N=75).

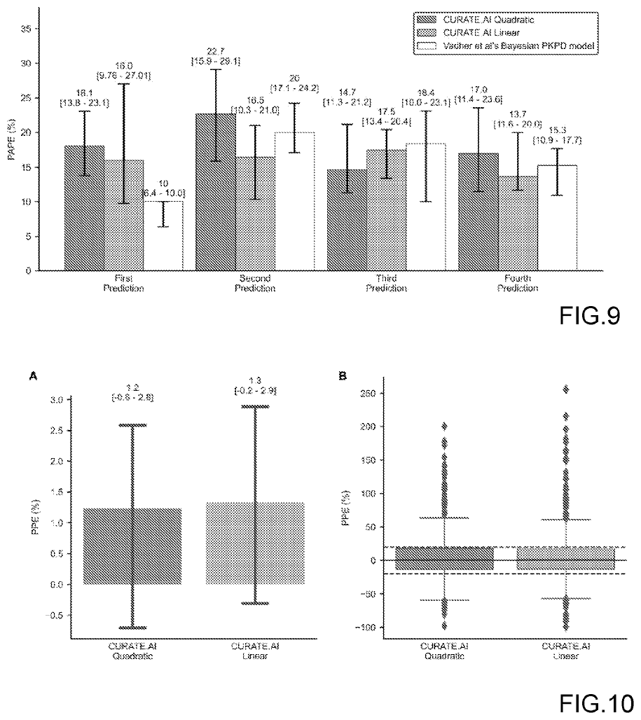

FIG. 9 is a chart showing Serial Percentage Absolute Prediction Error (PAPE) for dose recommendation methods. With Vadher's Bayesian PKPD model, the median PAPE was worse after the first prediction and improved in subsequent predictions and the day-to-day differences in PAPE were reported to be statistically significant. Conversely, the day-to-day differences in PAPE for CURATE.AI Quadratic and CURATE.AI Linear were not statistically significant.

The median PAPE for CURATE.AI Quadratic and Linear were also similar (Quadratic: 17.0%, Linear: 13.7%) with Vadher et al's model (15.3%) for the fourth prediction and this is when the Vadher et al's model was also expected to perform the best.

FIG. 9 shows Serial Percentage Absolute Prediction Error (PAPE). Median PAPE for the first 4 serial INR predictions for CURATE.AI Quadratic (N=62) CURATE.AI Linear (N=75) and Vadher et al's Bayesian PKPD model (N=74). The error bar represents 95% confidence interval. Comparison rests on the assumption that the underlying samples validated for each model are representative of a sample of patient population. N=number of patients.

### 2.5.3.2 PPE and PPE 20%

The median PPE results are shown in FIG. 8A. The median PPE across all predictions generated by CURATE.AI Quadratic (n=777) and CURATE.AI Linear (n=1016) was 1.2 and 1.3, respectively, with negligible bias.

The PPE 20% results for CURATE.AI Quadratic and Linear are shown in Table 4 and the distribution box plots are shown in FIG. 8B. 61.0% (n=474) and 62.3% (n=633) of INR predictions generated by CURATE.AI Quadratic and CURATE.AI Linear respectively were considered ideal (INR prediction within +20%). Both CURATE.AI models fared better in their predictive performance compared to Xue et al's dose-response model (57.9% ideal INR predictions), Xue et al's PKPD model (29.0% ideal INR predictions) and Hamberg et al's PKPD model (50.7% ideal INR predictions), while using less parameters (Table 4).

TABLE 4
INR PPE 20% from CURATE.AI and Literature (Number (Percentage)).
Categories (under prediction, ideal prediction and over prediction)
are based on a 20% threshold determined from literature.
Comparisons between CURATE.AI and Xue et al's dose-response
model, PKPD model and Hamberg et al's PKPD model rests
on the assumption that the underlying samples validated for
each model are representative of a sample of patient population.
n = total number of prediction events.

| | Under | Ideal | Over |
|---|---|---|---|
| Model | Prediction | Prediction | prediction |
| CURATE.AI Quadratic | 122 (15.7%) | 474 (61.0%) | 181 (23.3%) |
| (n = 777) | | | |
| CURATE.AI Linear | 156 (15.4%) | 633 (62.3%) | 227 (22.3%) |
| (n = 1016) | | | |
| Xue et al | 532 (20.2%) | 1525 (57.9%) | 576 (21.9%) |
| Xue et al | | | |
| Dose-response model | | | |
| Xue et al PKPD | 1390 (52.8%) | 764 (29.0%) | 479 (18.2%) |
| model | | | |
| Hamberg et al PKPD | 714 (27.1%) | 1336 (50.7%) | 583 (22.1%) |
| model | | | |

FIG. 10A and FIG. 10B are graphs comparing linear and quadratic methods. FIG. 10A and FIG. 10B show Percentage Prediction Error (PPE) (A): Bar plots. INR PPE for both CURATE.AI Quadratic (n=777) and Linear (n=1016). Color bars represent the median. Error bar represents 95% CI. (B): Box Plots. INR PPE for both CURATE.AI Quadratic (n=777) and Linear (n=1016). The lower bar and upper bar are the 2.5th and 97.5th percentile lines respectively. Dashed lines represent the +20% threshold for ideal predictions. n=total number of prediction events.

### 2.6 CURATE.AI Clinically Relevant Performance Metrics

### 2.6.1 Clinical Prediction Error (CPE)

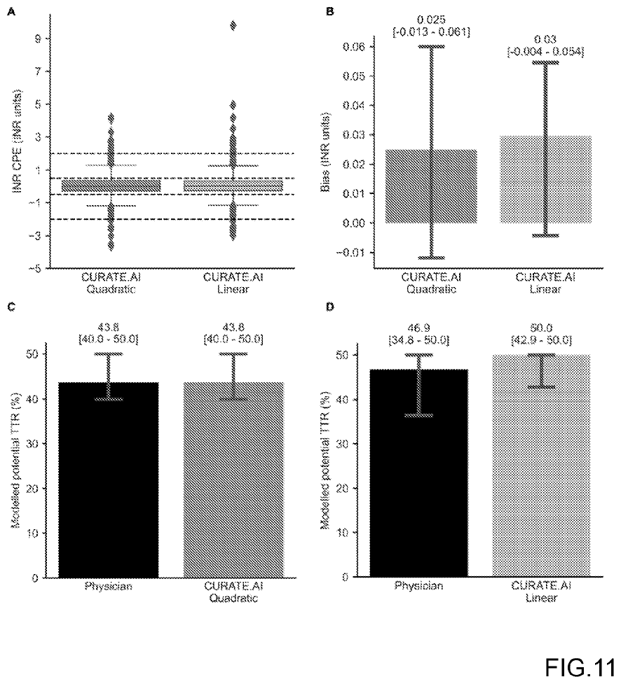

The CPE results for CURATE.AI are shown in Table 6. For the CPE threshold of 2.0, 97.0% (n=754) and 96.7% (n=982) of predictions from CURATE.AI Quadratic and Linear were considered ideal, respectively. For the CPE threshold of 0.5, 66.3% (n=515) and 68.0% (n=691) of the predictions were considered ideal (Table 6 and FIG. 11A).

TABLE 6
Clinical Prediction Error from Internal Evaluation of CURATE.AI
(Number (Percentage)). Categories (under prediction, ideal
prediction and over prediction) are based on 2.0 and 0.5
unit threshold. n = total number of prediction events.

| | | Under | Ideal | Over |
|---|---|---|---|---|
| Threshold | Model | Prediction | Prediction | prediction |
| 2.0 | CURATE.AI | 8 (1.03%) | 754 (97.0%) | 15 (1.93%) |
| | Quadratic | | | |
| | (n = 777) | | | |
| | CURATE.AI | 11 (1.08%) | 982 (96.7%) | 23 (2.26%) |
| | Linear | | | |
| | (n = 1016) | | | |
| 0.5 | CURATE.AI | 114 (14.7%) | 515 (66.3%) | 148 (19.0%) |
| | Quadratic | | | |
| | (n = 777) | | | |
| | CURATE.AI | 142 (14.0%) | 691 (68.0%) | 183 (18.0%) |
| | Linear | | | |
| | (n = 1016) | | | |

### 2.6.2 Underprediction Bias

The bias for both CURATE.AI Quadratic and CURATE.AI Linear are shown in FIG. 9B. The median bias for both CURATE.AI Quadratic and CURATE.AI Linear were 0.025 and 0.03, respectively, and the upper and lower bound values for the 95% confidence interval were clinically insignificant. This suggests that CURATE.AI predictions resulted in negligible underprediction bias.

### 2.6.3 Modelled Potential Time in Therapeutic Range (TTR)

The bar plots of modelled potential TTR for both CURATE.AI Quadratic and CURATE.AI Linear against physician-guided dosing are shown in FIG. 9C and FIG. 9D, respectively. Differences in modelled potential TTR between CURATE.AI Quadratic and physician-guided dosing and between CURATE.AI Linear and physician-guided dosing were not statistically significant.

FIG. 11A to FIG. 11D are graphs a comparison of dosing recommendations generated using methods of embodiments of the present invention with physician guided dosing. FIG. 11A to FIG. 11D show (A) INR Clinical Prediction Error (CPE), (B) Bias (INR units), and (C) (D) Modelled Potential TTR (%). CPE, in terms of INR units for both CURATE.AI Quadratic (n=614) and CURATE.AI Linear (n=774). The lower bar and upper bar are the 2.5th and 97.5th percentile lines respectively. Dashed lines represent the +2.0 and +0.5 threshold for ideal predictions. Bias, expressed as prediction error for CURATE.AI Quadratic (n=614) and CURATE.AI Linear (n=774). The lower bar and upper bar represent the lower and upper range of 95% CI respectively. Modelled potential TTR expressed as percentage, for both (C) CURATE.AI Quadratic (N=91) and (D) CURATE.AI Linear (N=118) against physician-guided dosing. The error bar represents the lower and upper range of 95% confidence interval, respectively. Normality testing performed using Shapiro-Wilk at 0.05 level of significance. Statistical comparison was performed with Wilcoxon signed rank test at 0.05 level of significance. *p<0.05. N=number of patients. n=total number of prediction events

### 2.7 Comparison Between CURATE.AI Quadratic and CURATE.AI Linear

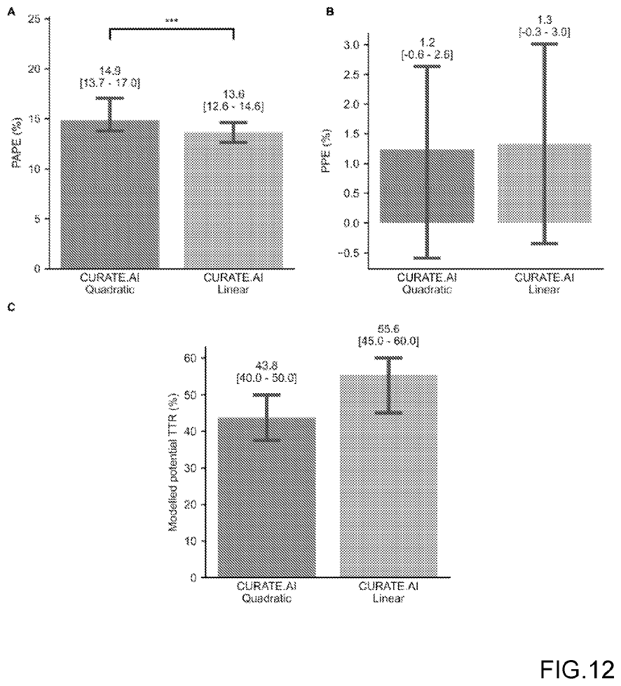

Beyond exploring the conventional second-order polynomial CURATE.AI Quadratic, this analysis also explored first-order variants of CURATE.AI. FIG. 12A to FIG. 12C are graphs showing comparisons of quadratic and linear methods.

Pairwise comparison between CURATE.AI Quadratic and CURATE.AI Linear for the same prediction events (n=777) across both technical performance and clinical relevance are shown in FIG. 12. CURATE.AI Linear's predictions are significantly more accurate compared to CURATE.AI Quadratic (P<0.001) (FIG. 12A). No differences were observed for bias metrics percentage PE and modelled potential TTR (FIG. 12B, FIG. 12C).

FIG. 12A to FIG. 12C show Bar Plots of Precision, Bias and Modelled Potential TTR Comparing Between CURATE.AI Quadratic and Linear (%). (A): PAPE for both CURATE.AI Quadratic and Linear (n=777). (B): PPE for both CURATE.AI Quadratic and Linear (n=777) (C): Modelled potential TTR (%) for CURATE.AI Quadratic and Linear (N=91). The lower bar and upper error bar represents 95% confidence interval. Comparison rests on the assumption that the underlying samples validated for each model are representative of a sample of patient population. No statistically significant difference was detected between the conditions t with Wilcoxon signed rank test at α=0.05. *** p<0.001. N=number of patients. n=total number of prediction events.

### 2.7.1 Five-Fold Cross Validation

Five-fold cross validation results for the evaluation of both CURATE.AI Quadratic and Linear for PAPE are shown in Table 7. The Median PAPE for both the training set and test sets were similar at 15.19+0.46 and 15.46±1.35, respectively, for CURATE.AI Quadratic; and 14.3±0.16 and 14.37±0.69, respectively, for CURATE.AI Linear. This suggests that factors such as patient demographics, clinical characteristics and genetic factors present in the used data set do not affect the predictive performance of both CURATE.AI Quadratic and CURATE.AI Linear and that the CURATE.AI models may be generalizable to other data sets of shared characteristics.

The retrospective analysis of warfarin data demonstrates how CURATE.AI can be assessed beyond technical performance, but also for clinical relevance thanks to its adaptability.

### CURATE.AI-Assisted Dose Titration for Anti-Hypertensive Personalised Therapy (CURATE.AI-ADAPT)

Hypertension is a serious public health concern, and the leading attributable risk factor for death and disability worldwide. Despite a wide array of effective medications, there remain significant challenges to utilizing these proven drugs to their highest potential. First, physicians frequently adopt a stepped-care approach in conventional drug selection and dose escalation strategies. Specifically, treatment drugs—in monotherapies, or two-drug combinations—are typically initiated at low doses and subsequently up-titrated. However, if limited efficacy or poor tolerance is observed, patients will be switched to other drugs, and this process repeats until the patients respond. Currently, the control rates among known hypertensive patients (defined as blood pressure (BP) below 140/90 mmHg) remain low: averaging about 21% worldwide, and ranging from 10% to 50% across countries. This sub-optimal outcome is often due to the intra- and inter-patient variability that causes patients to respond differently to the same treatment at different time points. This problem is further exacerbated in multi-drug therapy as drug-drug interactions are dose-, time-, and patient-dependent, and therefore, highly unpredictable. Patients can be perceived as non-responders to therapy that may otherwise work when given at the correct doses and time points. Secondly, current treatment decisions are guided by office BP measurements at a single timepoint or every three to twelve months, which may not be sufficient. Alternatively, out-of-office BP measurements (e.g., home BP), have been recommended as important adjuncts for hypertension diagnosis and prognosis. However, their frequent use for longitudinal monitoring and treatment guidance is still limited.

With rapid technological advancements, digital health innovations (DHIs) hold potential to overcome these issues. In particular, wearable sensors and telemonitoring platforms present an opportunity for reliable treatment monitoring as physicians gain access to out-of-office BP data. Mobile health (mHealth) apps incorporating strategies such as education, drug adherence reminder and behavioral counselling could complement the pharmaceutical interventions toward improving BP control. While these existing BP devices and digital management systems have yet to be widely adopted in standard clinical practice, many of them have already been undergoing rigorous clinical trials. Furthermore, emerging DHIs have also bolstered enthusiasm for ‘big data’ approaches that require a vast amount of population and patient-specific data to predict treatment responses and guide drug and dose selection. Although using big data for personalized care is promising, there remain persistent concerns over their practical implementation and true personalization outcomes in clinics.

For these reasons, there has been growing appreciation for ‘small data’ or N-of-1 approaches with promising potential for implementation and accurate outcomes. CURATE.AI is an Artificial Intelligence-driven clinical decision support system (AI-driven CDSS) that relies on this approach, and is designed to assist clinicians in identifying personalized doses for titration along the individual patient's treatment course. CURATE.AI requires only patient-specific data, such as doses (input) and BP responses (phenotypic output), to systematically investigate the patient's response to a drug and dose range, and dynamically identify personalized doses along the treatment course. Due to its ‘N-of-1’ or patient-specific workflow, CURATE.AI does not require population data to train AI models to subsequently dose individual patients. Instead, it uses only a patient's own data to prospectively calibrate their unique response to treatment. This patient-specific small dataset is then used to guide only the patient's own care. This is a critical differentiator of CURATE.AI from traditional AI models and approaches. Therefore, due to its relatively modest resource requirements compared to conventional big data AI as well as prior validation, CURATE.AI holds promise to improve patient outcomes, while avoiding the high-burden information pitfall of complex AI models.

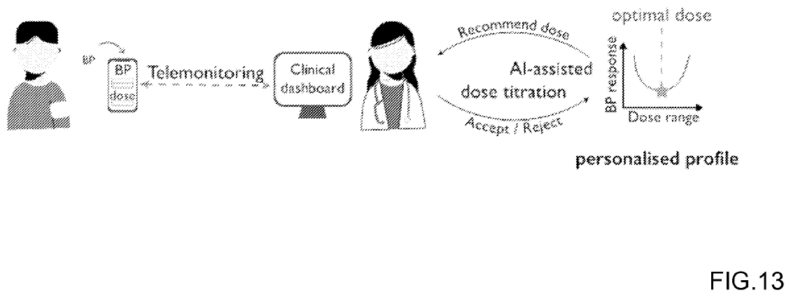

FIG. 13 shows an application of the dose recommendation system to a mobile health platform. In this protocol, we describe the CURATE.AI-assisted dose titration intervention which comprises two components-daily home BP monitoring via a telemonitoring platform; and personalized continuous dose titration with CURATE.AI recommending doses to physicians to assist them in dose titration.

All participants will be prescribed one month of two-drug combination therapy (dihydropyridine CCB+ARB/ACE-i) and will subsequently follow treatment schedules. Two BP readings, one minute apart, will be obtained with the provided standard home BP device. Measurements will be done in seated position, after two minutes of rest and at least 30 minutes after smoking, consuming alcohol, and exercising, within one hour of waking up, after urination, and before taking food and medication. Treating physicians may titrate participants' drug doses based on home BP according to SOC. Dose recommendations by CURATE.AI will be provided to the physicians for each participant for profile generation.

CURATE.AI will receive the data after their de-identification by the trial coordinator. The dataset will include daily dose adherence (i.e, whether the participant reports taking or skipping the prescribed dose on the telemonitoring platform) and home BP readings. After a profile is generated, participants will move into Stage 2, during which dose recommendations by CURATE.AI will be provided to the physicians for each participant with efficacy-driven intent. The physicians will make the final dose decision, as described above. All dose titrations will be communicated to the participants in-person or via a telemonitoring platform, text messages or calls which are cybersecurity and patient-privacy compliant. Dose titration will be done up to once per cycle, during which participants will take the drug dose as prescribed.

### Telemonitoring Platform

BotMD Care—a telemonitoring platform for capturing participant treatment data (e.g. BP, heart rate, measurement time, dose adherence)—will be used. Participants can access BotMD Care via common chat platforms such as Whatsapp, and will be provided with a secured link to report home monitoring data. Data will be securely transferred and compiled on a BotMD Care Clinical Dashboard for physicians' access. BotMD Care is currently deployed to manage clinically and technically suitable patients with hypertension, allowing patients to report their BPs, and physicians to monitor their patients' BPs remotely.

### CURATE.AI Internal Workflow

### Stage 1: Calibration-Intent Doses to Generate Personalized Dose-Response Profile

At least three dose levels and corresponding BP responses (or their mathematical transformations) from each participant are required to generate a quadratic CURATE.AI personalized profile. Thus, participants will first undergo a data collection stage for the purpose of generating a CURATE.AI profile. CURATE.AI will recommend a set of three calibration-intent doses of the selected drug for consideration by the treating physician(s). Corresponding BP responses will be collected. The CURATE.AI team will analyse all collected data to determine whether a profile can be generated. The preceding steps may be repeated to obtain an actionable CURATE.AI profile before proceeding to Stage 2.

### Stage 2: Efficacy-Driven Doses

Participants with actionable CURATE.AI profiles will enter Stage 2, where recommended doses will aim to achieve the target home BPs, defined as home SBP<135 mmHg and/or home DBP<85 mmHg. When the participants' BPs are outside the target range, CURATE.AI will recommend to the physicians an efficacy-driven dose based on the generated profile. If the target BPs are already achieved, CURATE.AI may not recommend any dose adjustment. In both cases, the physicians have the final decision on the prescribed doses. Participants will continue with HBPM under careful telemonitoring by the physicians throughout the treatment duration. All new data collected will be analyzed to determine whether the CURATE.AI profiles are reliable to be used for efficacy-driven dose recommendation. In the presence of systemic changes that may affect the BP responses across dose range, such as addition/removal of anti-hypertensive drugs and hospitalization events, a profile recalibration (i.e., creating a new profile or adjusting the existing profile) may be required. In such case, the participant may need to re-enter Stage 1.

A Retrospective Study of Artificial Intelligence-derived Personalized Tacrolimus Dosing for Pediatric Liver Transplant will now be described. Tacrolimus is the cornerstone of immunosuppressive therapy after pediatric liver transplantation. However, reliance on the physician's experience for dose titration, coupled with tacrolimus's narrow therapeutic window and inter and intra-patient variability, often results in frequent under or over-dosing events with detrimental patient outcomes. Existing predictive dose personalization models are not readily feasible for clinical implementation, as they require multiple measurements each day while the standard frequency is once daily. We developed CURATE.AI, a small-data artificial intelligence-derived platform, as a clinical decision support system to dynamically personalize doses using the patient's own data obtained once a day. Retrospective dose personalization with CURATE.AI on 16 patients' data demonstrated potential to enable more patients to reach therapeutic range within the first week. Our findings support the testing of CURATE.AI in a prospective controlled trial as an aid for the physician's decision on tacrolimus dose personalization after pediatric liver transplantation.

Liver transplantation is an established treatment for children with decompensated liver disease, liver-based metabolic disorders, acute liver failure, and unresectable primary liver malignancy. Continued improvements in surgical techniques, peri-operative care, and immunosuppression over the last 40 years have enabled a 5-year survival rate of over 85% after pediatric liver transplantation. These resulted in the shift of focus towards reducing the immunosuppression-associated morbidity (including neurotoxicity and nephrotoxicity) by minimizing doses and balancing with the risks of acute or chronic liver rejection as a consequence of minimized immunosuppression.

Tacrolimus is the most common immunosuppressive agent utilized in pediatric liver transplantation. The current standard of care (SOC) for post-transplant immunosuppression is personalized empirical tacrolimus dose adjustments based on tacrolimus trough levels (TTL). The personalized dose adjustments are guided as per the experience of the transplant physician. However, tacrolimus has a narrow therapeutic window and large inter- and intra-subject pharmacokinetic variability. As a result, SOC dose selection results in frequent deviations from the target therapeutic range, increasing the risk of clinically significant adverse effects due to under or over-immunosuppression. With a greater cumulative exposure to immunosuppressive agents that occurs throughout the patients' lifetime—thereby increasing the risk of associated morbidity—there is a dire need for optimal immunosuppression in pediatric liver transplantation.

CURATE.AI is a mechanism-independent and indication-agnostic artificial intelligence (AI)-derived dose optimization platform that only uses an individual patient's data of drug dose and phenotypic treatment response to calibrate a personalized response profile. It identifies and then recommends an optimal dose based on the individual's personalized profile to achieve the target treatment response for the patient. The profile of phenotypic responses across a dose range is represented as a smooth second-order surface. This second-order relationship was previously identified through neural network analysis, and subsequently experimentally validated for various indications. Of note, lower order polynomials of the first order or higher order polynomials may also be applicable depending on the intervention and indication. In its current form, implementing this platform is markedly simpler: without the need for neural network analysis and with high clinical actionability.

CURATE.AI requires two components to be supplied into the platform as inputs: the administered tacrolimus dose in mg (dose) and the corresponding TTL in ng/ml (response) to generate a personalized profile for each patient. While the used records showed the doses had been administered twice a day (BID), the dose input for CURATE.AI was the computed effective 24-hour dose. The response input was the corresponding TTL response measured the next day, right before the morning dose. Days with two measurements of TTL (taken in the morning and evening) instead of one, which is the standard frequency, were excluded. TTL measurements <2 ng/ml were assessed to be inaccurate as the measurement equipment was unable to provide resolution for TTL<2 ng/ml, and were thus excluded. All measurements in the records were only taken once each. Doses recorded from day 30 post-transplant onwards were not included as part of this study since it is during the initial post-transplant period that fluctuations in TTL are common; thereby requiring frequent adjustments in drug dosing. Hence, our study focused on the 30-day period following pediatric liver transplantation, which in most cases, matched with the timing of the discharge from the hospital. Doses recorded from the day of discharge onwards were assessed to be unreliable, as confirmation of the doses administered was not possible, and thus excluded. This study used dose-response pairs over a set of consecutive days for each patient for the analysis by CURATE.AI. Dose ranges were categorized as low (<2 mg), medium (2 mg to <4 mg), and high (≥4 mg).

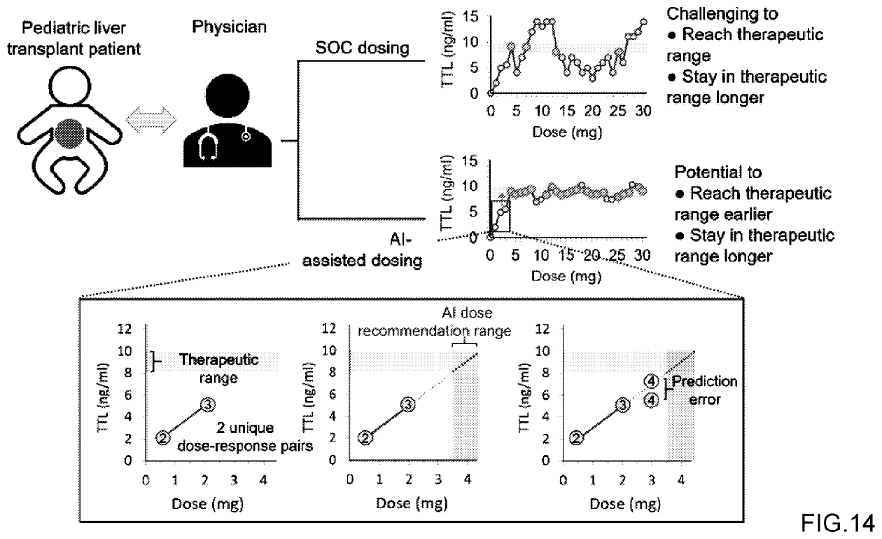

FIG. 14 illustrates a retrospective analysis of pediatric liver transplant data. Data from pediatric liver transplant patients were collected for the duration of 30 days post-transplant and were retrospectively analyzed. SOC dose selection relies on the unaided decisions by the physicians, with challenges to reaching the therapeutic range fast and staying within it. CURATE.AI-assisted dose selection aims to enable the TTL to reach the therapeutic range earlier and stay in the therapeutic range longer. The enlarged section below depicts an example of a dose-response relationship, also known as the individualized profile, based on the 2nd and 3rd days post-transplant. CURATE.AI-assisted dose recommendation for the 4th day is based on the intersection between the profile and the therapeutic range. A prediction of the TTL after the next dosing event was made based on the patient's CURATE.AI profile, and the prediction error was defined as the difference between the observed and predicted TTL.

The flow of the analysis with CURATE.AI is illustrated in FIG. 14. A linear equation was used to model the relationship between the tacrolimus dose and the corresponding TTL (response) and formed the individualized profile of the patient. Only 2 dose-response pairs were required for the linear equation to provide an individualized profile. The profile, which consisted of 2 dose-response pairs from 2 dosing events, was used to predict the TTL of the next dosing event. The difference between the observed TTL and the predicted TTL was defined as the prediction error.

Only 2 dose-response pairs were used as a calibration to provide a dose recommendation for the next dosing event. The choice of the 2 pairs required using a rolling window approach—defined as using a window of a specified size of 2 pairs that rolls through the data, 1 unit at a time for each profile generation. The most recent 2 data pairs with unique doses from prior to the timepoint that required a prediction and dose recommendation are used. This means that dose recommendation could only start after 2 dose-response pairs were obtained, requiring at least 2 days with unique doses and the corresponding TTL.

The Pearson correlation coefficient between predicted and observed TTL was computed. Additionally, CURATE.AI's performance was evaluated based on technical performance metrics widely used in literature to evaluate predictive models. Clinically relevant performance metrics were devised to characterize the potential clinical actionability of CURATE.AI as a CDSS.

The technical performance metrics were based on prediction errors, defined as the differences between the observed TTL and the TTL value predicted by CURATE.AI. Specifically, the technical performance metrics used were prediction error, absolute prediction error, and root mean squared error (RMSE).

For clinically relevant performance metrics, contextual information on pediatric liver transplant immunosuppression was considered in devising the metrics. Specifically, the clinically acceptable prediction error was defined as between and inclusive of of −10.5 and 2 ng/ml, based on the clinically acceptable range of TTL of 6.5 to 12 ng/ml, and in consideration of the therapeutic range of between and inclusive of 7 and 10 ng/ml

The range of potential dose recommendations was identified as the dose range at the intersection of the profile and therapeutic range, defined as between and inclusive of 7 and 10 ng/ml for every patient. The CURTE.AI dose recommendation(s) was identified as doses in multiples of 0.5 mg (the smallest tacrolimus capsule available) within this range. When multiple possible doses fulfilled the requirements, the lowest dose was considered as the CURATE.AI recommendation. The numbers of predictions that satisfy the following 5 assessment criteria were computed in steps. The first 2 criteria focused on CURATE.AI's prediction of TTL with the administered doses, and the next 3 criteria focused on CURATE.AI's dose recommendations' ability to achieve the therapeutic range. The criteria were: 1) CURATE.AI profile reliability (considered reliable if CURATE.AI correctly predicted that TTL would fall within the therapeutic range, pre-defined as between and inclusive of 7 and 10 ng/ml, or non-therapeutic range), 2) CURATE.AI prediction accuracy (considered accurate if CURATE.AI predictions were within the range of clinically acceptable prediction error, pre-defined as between and inclusive of −1.5 and 2 ng/ml), 3) whether the CURATE.AI-recommended dose differed from the dose administered, 4) whether the observed TTL was outside of the therapeutic range, and 5) dose actionability of the CURATE.AI-recommended dose (considered actionable if CURATE.AI dose recommendations were 8 mg or below).

2 representative patient cases were investigated to identify and characterize potential scenarios in which using CURATE.AI could be beneficial. The projected effects of utilizing CURATE.AI on each day of the treatment were explored, and the effects of CURATE.AI were categorized into 3 groups, namely: ‘no effect’, ‘improve’, or ‘worsen’ the time within the therapeutic range. The effects are based on the 5 assessment criteria listed previously.

The normality of the data distribution was tested with the Shapiro-Wilk test. Continuous variables were represented as mean (standard deviation (SD)) or median (interquartile range (IQR)), depending on the normality of the data type. Two-sided Wilcoxon signed-rank test was used to compare the medians of two non-parametric groups. Two-sided paired t-test was used to compare the means of two related parametric groups. The statistical significance was defined as p<0.05. All analyses were performed in Python (version 3.9.13).

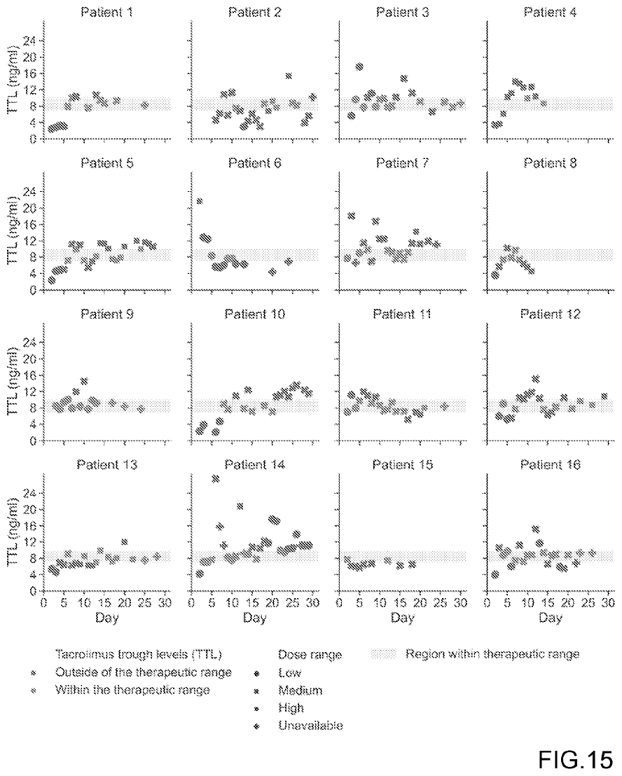

FIG. 15 shows TTL over time of each patient. The grey region represents the therapeutic range of between and inclusive of 7 and 10 ng/ml. Blue and orange markers represent TTL outside and within the therapeutic range, respectively. Dose ranges are categorized as low (<2 mg, circle markers), medium (2 mg to <4 mg, square markers), and high (≥4 mg, cross markers), respectively. Unavailable doses refer to missing or unreliable (from the day of discharge onwards) doses, and data points without TTL are not reflected here. Inter-individual heterogeneity was observed in terms of the dose ranges in which patients achieved the therapeutic range, the duration that patients stayed within the therapeutic range, and the number of days taken to first achieve the therapeutic range.

Inter-individual heterogeneity was observed in the longitudinal data (FIG. 2). Out of all 16 patients, 15 (93.75%) All patients achieved the therapeutic range at least for 1 day. The patients who achieved the therapeutic range did so at varied dose ranges: 2 (13.3312.50%) patients achieved the therapeutic range at low doses only, 3 4 (20.0025.00%) patients at medium doses only, 1 (6.67%) patient at high doses only, 4 (26.6725.00%) patients at both low and medium doses, 3 (20.0018.75%) patients at both medium and high doses, and 1 2 (6.6712.50%) patients across all dose ranges. The patients stayed within the therapeutic range for a mean of 42.18 23.94 (18.0015.29) % of days. The patients first achieved the therapeutic range within a median of 5.00 7.00 (IQR 4.50-8.002.73) days. Out of the 15 patients that achieved the therapeutic range for at least 1 day, 1013 (66.6781.25%) out of the 16 patients achieved the therapeutic range within the first week. Across all 16 patients, the mean TTL was 8.79 81 (3.2625) ng/ml (n=287 286 TTL) and the mean dose was 2.63 77 (1.4948) mg (n=276 331 doses), corresponding to a mean dose of 0.22 0.21 (0.170.16) mg/kg/day.

CURATE.AI demonstrated satisfactory predictive performance with a low median prediction error of 0.10 (IQR-1.75-2.20) ng/ml and an acceptable median absolute prediction error of 1.90 (IQR 0.80-3.20) ng/ml which is comparable to the mean absolute prediction error of the best-performing machine learning model by Song et al. that predicts TTL for infant liver transplant patients (mean: 2.01 ng/ml, mean+SD: 3.35 ng/ml, mean-SD: 0.85 ng/ml). The RMSE was 3.66 ng/ml for all predictions.

Reliable and accurate CURATE.AI profiles were generated for 23.93% of the days, indicating that CURATE.AI had the potential to identify doses for those dosing events to achieve the therapeutic range. The use of CURATE.AI to augment dose selection decisions, in turn, could lead to achieving a similar or higher percentage of days within the therapeutic range, as compared to the observed TTL in the data collected.

CURATE.AI-assisted dosing could have potentially achieved the therapeutic range on 10.26% more days, compared to the dosing events that achieved time within the therapeutic range with SOC dosing on the days for which predictions were made by CURATE.AI. For those days, CURATE.AI recommended slightly lower doses than SOC by a median of 0.50 (IQR-0.50-2.00) mg.

We investigated 2 representative patients' care which followed different dosing strategies to demonstrate clinical use cases where CURATE.AI may be beneficial.

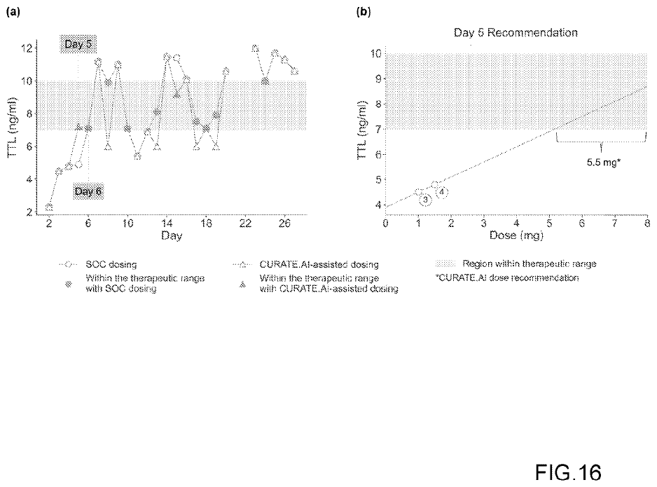

FIG. 16 shows a treatment journey for a patient with and without CURATE.AI. The grey region in both graphs represents the region within the therapeutic range, defined as between and inclusive of 7 and 10 ng/ml. (a) TTL achieved during SOC dosing (yellow circle markers without fill), TTL within the therapeutic range during SOC dosing (yellow circle markers with fill), projected TTL with CURATE.AI-assisted dosing (purple triangle markers without fill), projected TTL within the therapeutic range with CURATE.AI-assisted dosing (purple triangle markers with fill) are indicated. TTL on Days 21 and 22 were missing from the data collected. (b) Doses given on Days 2 and 3, leading to TTL on Days 3 and 4, are depicted in yellow circle markers without fill. The yellow points are numbered by the days on which the corresponding TTL was observed. CURATE.AI's dose recommendation of 5.5 mg would potentially enable achieving the therapeutic range on Day 5.

Patient 5 received a wide range of tacrolimus doses, from 0.5 to 5.0 mg, and achieved the therapeutic range only on Days 6, 8, 10, 13, 17, 18 and 19 (FIG. 16 a), out of the 26 days with available data.

For this patient, CURATE.AI successfully generated a profile from the 2 dose-response pairs from Days 3 and 4 (FIG. 16b). Based on the generated profile, CURATE.AI would have recommended 5.5 mg of tacrolimus (instead of the 2.0 mg dose administered) (FIG. 16b), which had the potential to lead to achieving the therapeutic range 1 day earlier, on Day 5 instead of Day 6 (FIG. 16a). Following that first CURATE.AI-assisted dosing event, the new data pair would have been incorporated back into the profile to ensure the profile evolves with the changes in the patient state and new dose recommendations can be generated over time. Based on the fact that a reliable CURATE.AI profile was obtained for 6 out of the next 15 consecutive days with complete data for this patient, and CURATE.AI suggested different doses than the administered doses on 4 of the 6 days, it is plausible that using CURATE.AI would not only have led the TTL to achieve the therapeutic range faster but also a higher percentage of days when the therapeutic range was achieved.

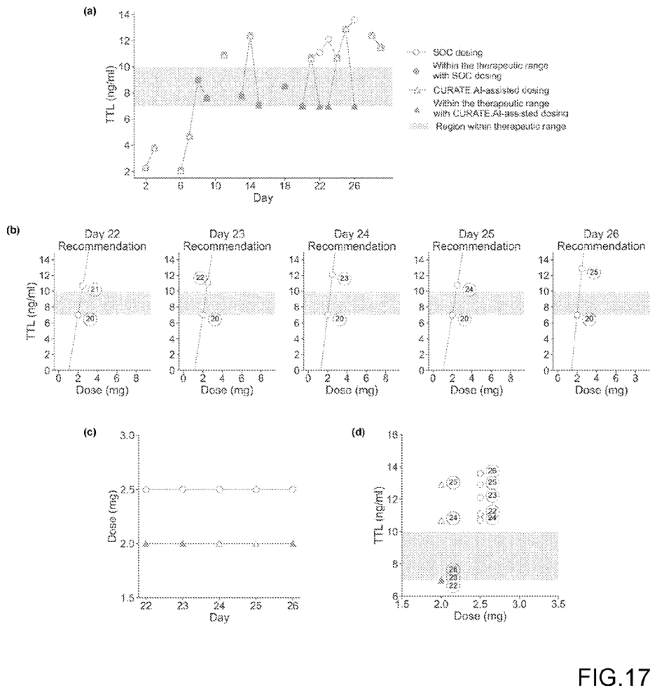

FIG. 17 shows CURATE.AI dose modulations to achieve and sustain the therapeutic range. (a) TTL achieved during SOC dosing (yellow circle markers without fill), TTL within the therapeutic range during SOC dosing (yellow circle markers with fill), projected TTL with CURATE.AI-assisted dosing (purple triangle markers without fill), projected TTL within the therapeutic range with CURATE.AI-assisted dosing (purple triangle markers with fill) are indicated. TTL on Day 13 was missing from the data collected. (b) Profiles generated for dose recommendations from Days 22 to 26 based on dose inputs of 2 mg on Day 19 and the corresponding TTL on Day 20 and the prior to the day of recommendation. (c) Doses administered over Days 22 to 26 (yellow circle markers) and the recommended doses by CURATE.AI (purple triangle markers) are indicated. (d) TTL corresponding to Days 22 to 26 (yellow circle markers) and the projected TTL with CURATE.AI dose recommendations (purple triangle markers) are indicated. The grey region spans the therapeutic range.

Patient 10 received 2 mg of tacrolimus on Day 19, which led to TTL within the therapeutic range on Day 20. Subsequently, the patient received 2.5 mg for 8 consecutive days, which led to TTLs outside the therapeutic range (TTL was unavailable on the 7th consecutive day on which 2.5 mg was received) (FIG. 7a). FIG. 7b illustrates the profiles generated for dose recommendations from Days 22 to 26 based on dose inputs of 4 mg on Day 3 and the day before the day of recommendation and the corresponding TTL. The slopes of the profiles varied in steepness over time with the same dose inputs of 2 mg and 2.5 mg, suggesting longitudinal variability in TTL response to tacrolimus dose. The dynamic TTL responses from the static and repeated doses over consecutive days suggest that the patient's state was changing, and repeatedly administering the same dose over consecutive days was not an optimal dosing strategy. With dose titrations recommended by CURATE.AI (FIG. 7b), the patient's TTL plausibly could have maintained within the therapeutic range for longer (FIG. 7a). The projected TTL, defined as the estimated TTL if CURATE.AI's actionable dose recommendations were administered, ranged from 8.55 to 14.00 ng/ml with CURATE.AI-assisted dosing for Days 22 to 26, compared to the observed TTL of 11.2 to 14.00 ng/ml which were all outside of the therapeutic range (FIG. 7c).

This retrospective study successfully generated profiles for 13 out of 16 patients using their individual data consisting of the recorded tacrolimus doses and the corresponding TTL over 30 days following liver transplantation. CURATE.AI demonstrated promising performance with both technical and clinically relevant performance metrics.

Other personalized tacrolimus dosing methods proposed for pediatric liver transplant are AUC-based methods that require resource-intensive, high-frequency measurements across the dosing interval and face the resource barrier for translation into clinical practice. CURATE.AI is less resource-intensive than any of the 13 machine learning models compared in the recently published study by Song et al. for personalized dosing for infants with liver transplants, as CURATE.AI requires only 2 parameters while the machine learning models required 3 to 7 parameters. Despite using a fraction of inputs, CURATE.AI achieved comparable technical performance-median 1.90 ng/ml absolute prediction error compared to mean 2.01 ng/ml absolute prediction error of the best-performing model described in the study.

Furthermore, CURATE.AI has the potential to overcome the issues of heterogeneity observed in the patient data. Specifically, the patients achieved the therapeutic range at different dose ranges and/or responded differently over time to the same dose (FIG. 17). The percentage of days within the therapeutic range and the number of days to achieve the therapeutic range also differed across patients. While it is noted that probabilistic models could account for uncertainties within empirical data, there are advantages of using CURATE.AI as it circumvents issues with big datasets and is mechanism-independent and indication-agnostic. In response to the aforementioned challenges, CURATE.AI used a personalized dosing approach by generating digital twins for individual patients, a concept that has demonstrated potential use for personalized treatments. CURATE.AI demonstrated the potential to identify the optimal doses to enable more patients to have their TTL reach the therapeutic range in the first week. Additionally, CURATE.AI could achieve similar results in terms of maintaining the TTL within the therapeutic range and the number of days required to for the TTL reach the therapeutic range.

These results suggest that CURATE.AI might be suitable for assisting tacrolimus dosing decisions in pediatric liver recipients to enable similar or better TTL, which may translate to improvement in outcomes.

FIG. 18 is a block diagram showing a technical architecture of a data processing system according to an embodiment of the present invention. Typically, the methods of dose recommendation and patient profile update according to embodiments of the present invention are implemented on a computer or a number of computers each having a data-processing unit. The block diagram as shown in FIG. 18 illustrates a technical architecture of a computer which is suitable for implementing one or more embodiments herein.

The technical architecture 1800 includes a processor 1822 (which may be referred to as a central processor unit or CPU) that is in communication with memory devices including secondary storage 1824 (such as disk drives), read only memory (ROM) 1826, random access memory (RAM) 1828. The processor 1822 may be implemented as one or more CPU chips. The technical architecture 1800 may further comprise input/output (I/O) devices 1830, and network connectivity devices 1832.

The secondary storage 1824 is typically comprised of one or more disk drives or tape drives and is used for non-volatile storage of data and as an over-flow data storage device if RAM is not large enough to hold all working data. Secondary storage 1824 may be used to store programs which are loaded into RAM 1828 when such programs are selected for execution. In this embodiment, the secondary storage 1824 has an input data receiving module 1802, a patient profile update module 1804, a dose recommendation module 1806, a decision tree module 1808, and an output module 1810 comprising non-transitory instructions operative by the processor 1822 to perform various operations of the methods of the present disclosure. As depicted in FIG. 18, the modules 1802-1810 are distinct modules which perform respective functions implemented by the data processing system 1800. It will be appreciated that the boundaries between these modules are exemplary only, and that alternative embodiments may merge modules or impose an alternative decomposition of functionality of modules. For example, the modules discussed herein may be decomposed into sub-modules to be executed as multiple computer processes, and, optionally, on multiple computers. Moreover, alternative embodiments may combine multiple instances of a particular module or sub-module. It will also be appreciated that, while a software implementation of the modules 1802-1810 is described herein, these may alternatively be implemented as one or more hardware modules (such as field-programmable gate array(s) or application-specific integrated circuit(s)) comprising circuitry which implements equivalent functionality to that implemented in software. The ROM 1826 is used to store instructions and perhaps data which are read during program execution. The secondary storage 1824, the RAM 1828, and/or the ROM 1826 may be referred to in some contexts as computer readable storage media and/or non-transitory computer readable media.

The I/O devices 1830 may include printers, video monitors, liquid crystal displays (LCDs), plasma displays, touch screen displays, keyboards, keypads, switches, dials, mice, track balls, voice recognizers, card readers, paper tape readers, or other well-known input devices.

The network connectivity devices 1832 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identity (RFID), and/or other air interface protocol radio transceiver cards, and other well-known network devices. These network connectivity devices 1832 may enable the processor 1822 to communicate with the Internet or one or more intranets. With such a network connection, it is contemplated that the processor might receive information from the network, or might output information to the network in the course of performing the method operations described herein. Such information, which is often represented as a sequence of instructions to be executed using processor 1822, may be received from and outputted to the network, for example, in the form of a computer data signal embodied in a carrier wave.

The processor 1822 executes instructions, codes, computer programs, scripts which it accesses from hard disk, floppy disk, optical disk (these various disk based systems may all be considered secondary storage 1824), flash drive, ROM 1826, RAM 1828, or the network connectivity devices 1832. While only one processor 1822 is shown, multiple processors may be present. Thus, while instructions may be discussed as executed by a processor, the instructions may be executed simultaneously, serially, or otherwise executed by one or multiple processors.

It is understood that by programming and/or loading executable instructions onto the technical architecture 1800, at least one of the CPU 1822, the RAM 1828, and the ROM 1826 are changed, transforming the technical architecture 1800 in part into a specific purpose machine or apparatus having the novel functionality taught by the present disclosure. It is fundamental to the electrical engineering and software engineering arts that functionality that can be implemented by loading executable software into a computer can be converted to a hardware implementation by well-known design rules.

Although the technical architecture is described with reference to a computer, it should be appreciated that the technical architecture may be formed by two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the technical architecture to provide the functionality of a number of servers that is not directly bound to the number of computers in the technical architecture. In an embodiment, the functionality disclosed above may be provided by executing the application and/or applications in a cloud computing environment. Cloud computing may comprise providing computing services via a network connection using dynamically scalable computing resources. A cloud computing environment may be established by an enterprise and/or may be hired on an as-needed basis from a third party provider.

The invention allows to personalize patient treatment based on small data, while incorporating their risk and AI-involvement preferences. The invention has prognostic and predictive capabilities to support early decision on the most effective treatment plan, and an opportunity to adjust it in the course of treatment, in response to the profile's shape changes. These properties unlock new capabilities in personalized treatment offering and estimate generation in new models of payment, e.g. outcome based payment, that are of value to payers: insurance agencies, public institutions and patients themselves.

Personalized precision dosing is increasingly appreciated as a viable alternative to the traditional dose escalation approach in hypertension management, to overcome the unpredictable inter- and intra-patient variability in treatment responses.AI-driven big data platforms have shown promise as tools to tailor dosing to subpopulations. However, small data approaches, such as CURATE.AI, may further enable a dynamic personalization of therapies at the individual level, which may be more suitable for long-term chronic disease management. CURATE.AI has been clinically validated across a wide range of disease indications, and has also shown promise in an earlier retrospective case series of its applicability for hypertension.6 The wide-ranging applicability of this platform may be attributed to its indication-agnostic and mechanism-independent properties.

1. A computer implemented method of generating a dose recommendation for a subject, the method comprising:
receiving subject data comprising an indication of a drug dose administered to the subject and an indication of a phenotypic response of the subject, the indication of the phenotypic response of the subject comprising an indication of a change in at least one phenotypic marker level;
updating a subject profile which models the phenotypic response of the subject to the drug dose; and
generating a dose recommendation for the subject from the subject profile.

2. The method according to claim 1, wherein the subject data comprises an indication of at least three drug doses administered to the subject and at least three corresponding phenotypic responses.

3. The method according to claim 1, wherein the dose recommendation for the subject is selected to calibrate the subject profile during a dynamic calibration phase.

4. The method according to claim 1, wherein generating the dose recommendation for the subject from the subject profile comprises transforming data from received subject data corresponding to different times.

5. The method according to claim 4, wherein transforming data from received subject data comprises determining a cumulative dose administered to the subject over a specified period of time.

6. The method according to claim 5, wherein the dose recommendation is generated taking into account the cumulative dose.

7. The method according to claim 6, wherein the dose recommendation is generated to account for minimal disruption in standard dosing protocol/workflow in standard of care in addition to accounting for unexpected dosing breaks.

8. The method according to claim 1, wherein generating the dose recommendation comprises determining a composite outcome measure which is dependent on a plurality of marker levels.

9. The method according to claim 1, wherein the subject profile is based on previously received subject data corresponding to a plurality of different times and the subject data is weighted according to the plurality of different times such that a higher weighting is given to more recent data points.

10. The method according to claim 1, further comprising receiving clinician inputs corresponding to a decision tree and wherein the dose recommendation is generated using the clinician inputs and the subject profile.

11. The method according to claim 10, wherein the decision tree is configured to generate a dose recommendation or generate an alert to clinicians for further actions.

12. The method according to claim 10, wherein the dose recommendation is generated with dual intents, the dual intents comprising dose selected using a generated subject profile that also fulfils an intent to update the subject profile.

13. The method according to claim 11, wherein generating an alert to clinicians for further actions comprises determining if the dose recommendation cannot be generated using the subject profile and the subject requires immediate clinical attention.

14. The method according to claim 1, wherein the dose recommendation is generated taking into account subject compliance and/or risk factors and/or risk preference.

15. The method according to claim 1, further comprising comparing the subject profile with their earlier profile or with profiles corresponding to different subjects.

16. The method according to claim 15, further comprising determining features of the subject profile that may reveal unexpected drug interactions, wherein the dose recommendation is generated taking into account the features of the subject profile.

17. The method according to claim 15, further comprising comparing the subject profile with profiles of a same subject from preceding timepoints to predict treatment outcomes over time.

18. The method according to claim 1, wherein the dose recommendation is selected to achieve a change in marker level to reach a target range/directional change or no change suiting needs of a treatment intent.

19. The method according to claim 1, further comprising selecting an algorithm or a combination of algorithms from a plurality of algorithms for updating the subject profile.

20. A data processing system configured to carry out a method according to claim 1.

21. A non-transitory computer readable carrier medium carrying processor executable instructions which when executed on a processor cause the processor to carry out a method according to claim 1.
